(19) United States
(12) Patent Application Publication
(10) Pub. No.: US 2022/0370823 A1
(43) Pub. Date: Nov. 24, 2022

(54) LIGHT BASED THERAPY HELMET

(71) Applicant: SANDER ELECTRONIC CO., LTD., Taipei (TW)

(72) Inventor: JIAJIN HUANG, TAIPEI (TW)

(21) Appl. No.: 17/328,198

(22) Filed: May 24, 2021

(51) Int. Cl. A61N 5/06; F21V 23/00; F21V 19/00; F21V 23/04; F21V 23/06

(52) U.S. Cl. CPC A61N 5/0617; F21V 23/008; F21V 19/0015; F21V 23/002; F21V 23/04; F21V 23/06; F21Y 2113/10

(57) ABSTRACT

A light based therapy helmet has a circuit board layer which is equipped with multiple lighting dots. Each of the multiple lighting dots respectively is a first, a second or a third light-emitting unit. The first, the second and the third light-emitting units respectively emit a light of a first, a second and a third light band which respectively include a wavelength of 660 nm, 940 nm and 1600 nm. A lower helmet body is provided with an accommodation space configured for a user's head to be accommodated directly. An upper helmet body is provided with an accommodation space that fits the circuit board layer. When the lower helmet body, the circuit board layer, and the upper helmet body fit together, the circuit board layer is fixed between the upper helmet body and the lower helmet body. A controller controls the on/off and the brightness of the multiple lighting dots.

### BACKGROUND OF THE INVENTION

### 1. Field of the Invention

The present invention relates to a therapy helmet, particularly to a light based therapy helmet.

### 2. Description of the Related Art

The prevention and treatment of hair loss and the promotion of healthy hair growth have long been the directions toward which the medical community's continuous efforts are pursuing. After the prevalence of laser technologies, the medical community tried to use low-dosage laser light sources to irradiate the scalp to prevent hair loss and promote healthy hair growth. Therefore, a medical laser hair growth device was developed. The medical community has also tried to use Light Emitting Diode (hereinafter referred to as LED) as a light source to illuminate the scalp to prevent hair loss and promote healthy hair growth. The above-mentioned phototherapy is effective for seborrheic alopecia, female postpartum alopecia, alopecia areata and hair loss after chemotherapy, etc. Moreover, the above-mentioned phototherapy can improve oil secretion, minimize the possibility of hair loss, prevent hair thickening, and make the hair more plumping and shiny. The above-mentioned phototherapy can also promote the hair to fully enter the growth phase, penetrate deep into the hair follicles, increase the number of hair follicles, and strengthen the hair roots.

Nowadays, devices in the market that utilize light to irradiate the scalp to prevent hair loss and promote healthy hair growth usually include far infrared light sources provided by far infrared light emissions from ceramic elements or particulate films after their heat absorption; nevertheless, the far infrared light sources may also be directly provided by the LEDs. However, there is still room for improvements for the above products in terms of the choices of the light source bands, the mechanical structure designs, and/or the manufacturing costs. Since more and more people have great enthusiasm for hair health, there are increasing urgent needs to design devices that are more effective in preventing hair loss and promoting healthy hair growth, along with better and more comfortable mechanical structures and more reasonable manufacturing costs.

### SUMMARY OF THE INVENTION

In order to meet the urgent needs, the present invention discloses a light based therapy helmet to improve the aforementioned light source band selections, mechanical structure designs, or/and manufacturing costs, so as to provide users with more comfortable, more effective, and more reasonably priced devices that could promote healthy hair growth. And the technical contents of the light based therapy helmet of the present invention include:

a circuit board layer, which includes: a circuit board; and multiple lighting dots, fixed on the circuit board; wherein each of the multiple lighting dots is respectively a first light-emitting unit, a second light-emitting unit or a third light-emitting unit, and the first light-emitting unit emits light of the first light band, and the second light-emitting unit emits light of the second light band, and the third light-emitting unit emits light of the third light band;

a lower helmet body, which includes: a body; and an accommodation space, configured for a user's head to be accommodated therein directly, wherein the accommodation space is defined by the body of the lower helmet body;

an upper helmet body, which includes: an accommodation space, which fits the circuit board layer and the lower helmet body;

when the lower helmet body, the circuit board layer, and the upper helmet body fit together, and the circuit board layer is fixed between the upper helmet body and the lower helmet body;

a controller, which controls the ON/OFF states and emission brightness of the multiple lighting dots;

a set of interface wires connected between the circuit board layer and the controller.

Preferably, a number of the first light-emitting unit, a number of the second light-emitting unit and a number of the third light-emitting unit are each no less than 24.

Preferably, the body of the lower helmet body is made of transparent materials, and locations of the first light-emitting unit, the second light-emitting unit and the third light-emitting unit on the circuit board layer are configured in such a way that the collection of the respective light emissions from all of the first light-emitting units, all of the second light-emitting units and all of the third light-emitting units passes the body of the lower helmet body respectively and shines uniformly.

Preferably, the light of the first light band, the light of the second light band and the light of the third light band are respectively red light, infrared light and far infrared light, and include respectively a wavelength of 660 nm, a wavelength of 940 nm and a wavelength of 1600 nm.

Preferably, the body of the lower helmet body is made of transparent materials, and the respective average transparencies of the transparent materials with respect to the light of the first light band, the light of the second light band and the light of the third light band are no less than 5%.

In one embodiment, the technical contents of the light based therapy helmet of the present invention further include:

a circuit board layer, which includes: a circuit board; multiple lighting dots, which are fixed on the circuit board, wherein each of the multiple lighting dots is a set of light-emitting units, and the set of light-emitting unit includes a first LED which emits light of a first light band, a second LED which emits light of a second light band and a third LED which emits light of a third light band.

Preferably, the number of the multiple lighting dots is no less than 72.

Preferably, the body of the lower helmet body is made of transparent materials, and locations of the multiple lighting dots on the circuit board layer are configured in such a way that the collection of the light emissions from all of the multiple lighting dots passes the body of the lower helmet body and shines uniformly.

Preferably, the body of the lower helmet body has multiple through holes, and the light emitted by each of the multiple lighting dots passes through a respective one among the multiple through holes, and the locations of the multiple lighting dots on the circuit board layer are configured in such a way that the collection of light emitted by the multiple lighting dots passes the multiple through holes and shines uniformly.

Preferably, the controller has at least one on-off button to turn on/off the multiple lighting dots on the circuit board layer and at least one brightness control button to control the brightness of the collection of light emitted by the multiple lighting dots.

Preferably, the controller further includes: a power input end to receive power from an external power source.

Preferably, the controller transmits a set of control signals to the circuit board layer through the set of interface wires, so as to supply electric power to the multiple lighting dots, and to control the brightness of the collection of light emitted by the multiple lighting dots.

Preferably, the type of connections configured among the circuit board layer, the set of interface wires and the controller is compatible with Universal Serial Bus Type-C cables and connectors.

Preferably, the shape of the circuit board layer conforms to the shapes of the upper helmet body and the lower helmet body, and the lower helmet body has a bottom edge slot which integrates with the circuit board layer to fix the circuit board layer on the lower helmet body.

Preferably, the lower helmet body further includes multiple cushions which are fixed on the inner surface of the body of the lower helmet body.

Preferably, the multiple cushions are made of plastic, leather, Styrofoam, sponge, foam, paper, bamboo, rattan, grass, wood, metal or rubber.

### BRIEF DESCRIPTION OF THE DRAWINGS

FIG. 1 is a perspective view of a light based therapy helmet of the present invention;

FIG. 2 is an exploded view of a light based therapy helmet of the present invention;

FIG. 3 is an inner side perspective view of the light based therapy helmet of the present invention;

FIG. 4 is another inner side perspective view of the light based therapy helmet of the present invention.

### DETAILED DESCRIPTION OF THE INVENTION

In the following, the technical solutions in the embodiments of the present invention will be clearly and fully described with reference to the drawings in the embodiments of the present invention. Obviously, the described embodiments are only a part of the embodiments of the present invention, but not all the embodiments. Based on the embodiments of the present invention, all other embodiments obtained by a person of ordinary skill in the art without creative efforts shall fall within the protection scope of the present invention.

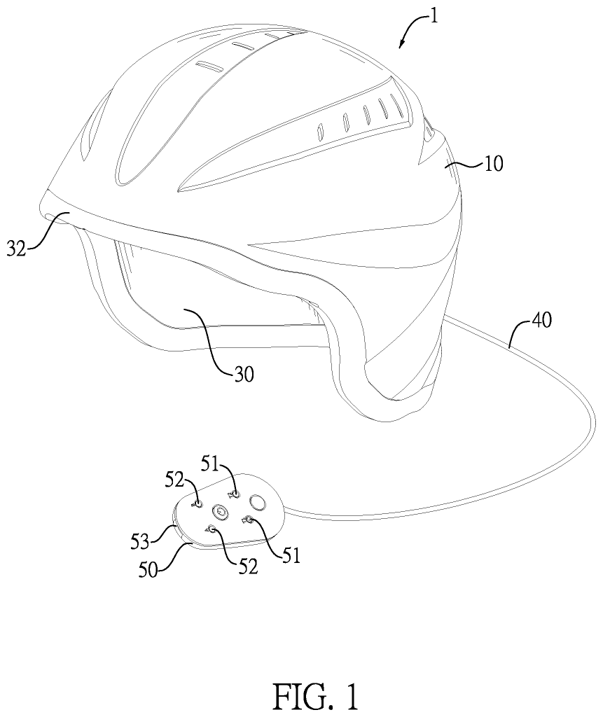

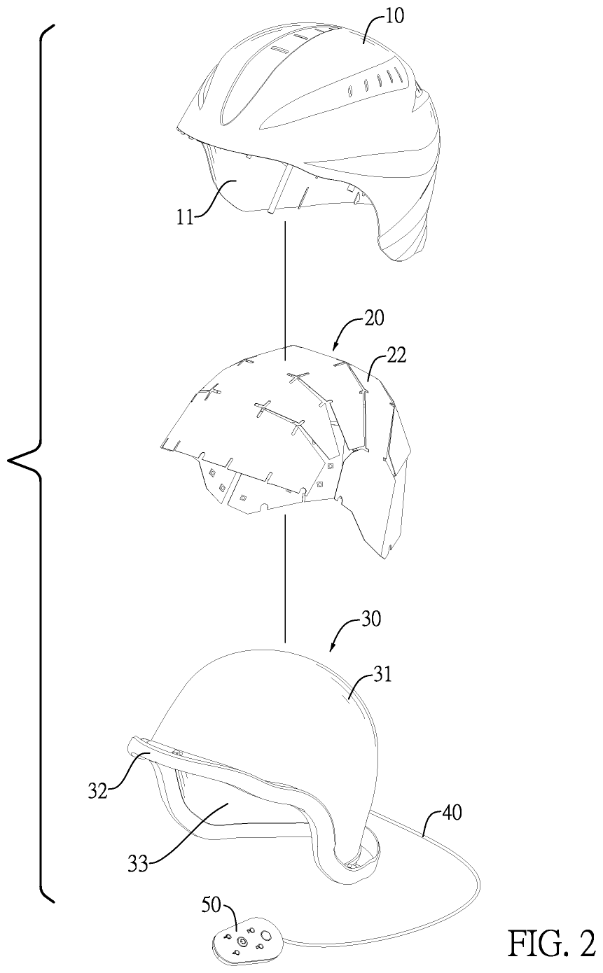

Please refer to FIG. 1 and FIG. 2. FIG. 1 is a perspective view of a light based therapy helmet of the present invention and FIG. 2 is an exploded view of the light based therapy helmet of the present invention. The light based therapy helmet 1 includes an upper helmet body 10, a circuit board layer 20, a lower helmet body 30, a set of interface wires 40, and a controller 50. The light based therapy helmet 1 is for users to wear on their heads. When a user wears the light based therapy helmet 1 on his/her head, the controller 50 will control the circuit board layer 20 of the light based therapy helmet 1 to emit light passing through the lower helmet body 30 and shining on the user's head so as to promote healthy hair growth.

The upper helmet body 10 is the outer casing of the light based therapy helmet 1, the upper helmet body 10 has an accommodation space 11, and the upper helmet body 10 is fitted with the circuit board layer 20 and the lower helmet body 30 through the accommodation space 11 of the upper helmet body 10. The lower helmet body 30 has a body 31, a bottom edge slot 32 and an accommodation space 33 defined by the body 31 of the lower helmet body 30. The accommodation space 33 is for a user to directly wear the light based therapy helmet 1 on the head. The shape of the circuit board layer 20 conforms to the shapes of the upper helmet body 10 and the lower helmet body 30. When the upper helmet body 10, the circuit board layer 20 and the lower helmet body 30 are fitted together, the circuit board layer 20 is firmly fixed between the upper helmet body 10 and the lower helmet body 30. In this way, the upper helmet body 10, the circuit board layer 20 and the lower helmet body 30 are modularized and can be manufactured independently first and then assembled together neatly to lower the production costs.

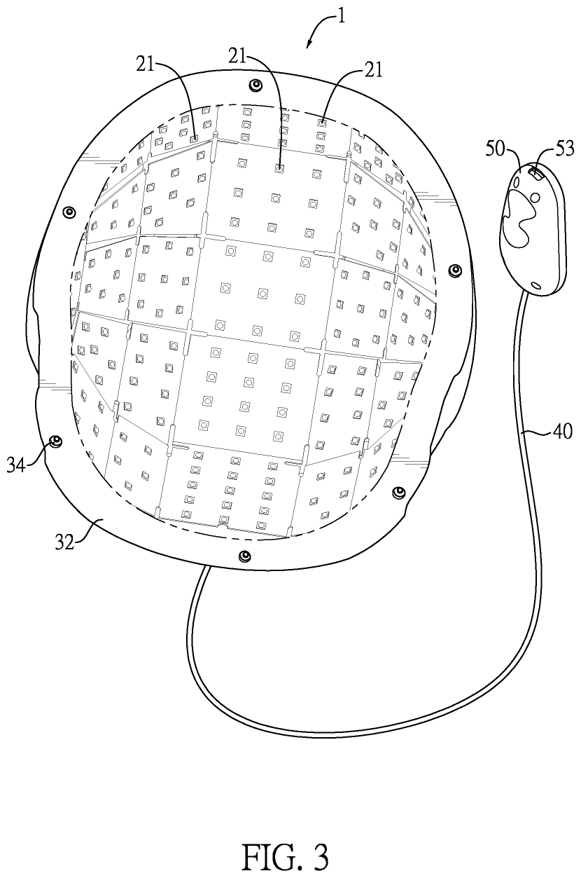

Please refer to FIG. 2 and FIG. 3. In FIG. 3, the body 31 of the lower helmet body 30, located in the dotted area, is not shown in FIG. 3. The circuit board layer 20 has multiple lighting dots 21 and a circuit board 22, with the multiple lighting dots 21 fixed on the circuit board 22 of the circuit board layer 20. The bottom edge slot 32 of the lower helmet body 30 has multiple screw holes 34, and the circuit board layer 20 is fixed firmly on the lower helmet body 30 through the multiple screw holes 34 of the bottom edge slot 32.

In one embodiment, the body 31 of the lower helmet body 30 is made of transparent materials, such as polymer light-transmitting materials, including: poly-methyl methacrylate (PMMA), polystyrene (PS), polycarbonate (PC), dially glycol carbonates (CR-39), styrene acrylonitrile copolymer (SAN), poly-4-methylpentene-1 (TPX), transparent polyamide, OZ-1000 resin, KT-153 spiro alkane resin, TS series optical materials, MH resin, APO resin and TS26 Resins. In addition, the transparent materials can also be polymer-based light-transmitting composite materials, including: transparent glass fiber reinforced plastic.

In one embodiment, the circuit board layer 20 has multiple lighting dots 21, each one of the multiple lighting dots 21 can be a first light-emitting unit, a second light-emitting unit, or a third light-emitting unit. The locations of the first light-emitting units on the circuit board layer 20 are configured in such a way that the collection of light emitted by all of the first light-emitting units passes through the body 31 of the lower helmet body 30 and shines uniformly on the surface of a user's head. Likewise, the locations of the second light-emitting units on the circuit board layer 20 are configured in such a way that the collection of light emitted by all of the second light-emitting units passes through the body 31 of the lower helmet body 30 and shines uniformly on the surface of a user's head. Similarly, the locations of the third light-emitting units on the circuit board layer 20 are configured in such a way that the collection of light emitted by all of the third light-emitting units passes through the body 31 of the lower helmet body 30 and shines uniformly on the surface of a user's head. Wherein, the first light-emitting unit, the second light-emitting unit and the third light-emitting unit all include an LED. The first light-emitting unit emits light of the first light band, the second light-emitting unit emits light of the second light band, and the third light-emitting unit emits light of the third light band. Preferably, the first light band emitted from the first light-emitting unit is red light and includes a wavelength of 660 nm; the second light band emitted from the second light-emitting unit is infrared light and includes a wavelength of 940 nm; the third light band emitted from the third light-emitting unit is far infrared light and includes a wavelength of 1600 nm. By mixing and shining the light of the first, the second and the third light bands on the cell tissue of the head, cells get activated; hair follicles get stimulated; blood circulation gets promoted; and atrophic damaged hair follicles get repaired, such that, the effects of promoting healthy hair growth and nourishing the scalp can be achieved. Therefore, the present invention has better effect for promoting healthy hair growth than those phototherapy products with single light band or dual light band in the present market. In addition to the above-mentioned health benefits on scalp of the present invention, the present invention also has the beneficial effects on keeping the head warm, improving the body health, and reducing body tremors and discomforts.

In one embodiment, the light of the first light band emitted by the first light-emitting unit, the light of the second light band emitted by the second light-emitting unit, and the light of the third light band emitted by the third light-emitting unit all have higher transmittances in terms of the transparent materials of the body 31 of the lower helmet body 30. For example, the transmittances are no less than 5%.

Preferably, the circuit board layer 20 has more than 24 first light-emitting units, 24 second light-emitting units and 24 third light-emitting units.

The two ends of the set of interface wires 40 are respectively connected with the controller 50 and the circuit board layer 20, wherein the controller 50 transmits electric power and a set of control signals to the circuit board layer 20 through the set of interface wires 40. The type of connections configured among the circuit board layer 20, the set of interface wires 40 and the controller 50 is compatible with Universal Serial Bus Type-C cables and connectors.

The controller 50 has at least one on-off button 51 to turn on/off all of the first light-emitting units, all of the second light-emitting units and all of the third light-emitting units on the circuit board layer 20. The controller 50 also has at least one brightness control button 52 to control the brightness of the collection of light from all of the first light-emitting units, all of the second light-emitting units and all of the third light-emitting units; wherein the controller 50 may use duty cycle to perform the aforesaid brightness control. Furthermore, the controller 50 has a power input end 53 to receive electric power from an external power source, such as a 5V/2Amax mobile power source or a 12V vehicle DC power source and so on. In this way, users may use the mobile power source or the vehicle DC power source to supply power to the light based therapy helmet 1 of the present invention when they wear the light based therapy helmet 1 of the present invention and engage in outdoor activities, such as biking, walking, riding on public transportation or riding on private vehicles without being restricted by power supply.

In one embodiment, the lower helmet body 30 is made of the aforesaid transparent materials. Each of the multiple lighting dots 21 of the circuit board layer 20 is a set of light-emitting units (not shown in drawings), wherein each one of the set of light-emitting units has a first LED, a second LED and a third LED, and the first LED emits light of the first light band which is red light and includes the wavelength of 660 nm, and the second LED emits light of the second light band which is infrared light and includes the wavelength of 940 nm, and the third LED emits light of the third light band which is far infrared light and includes the wavelength of 1600 nm. The locations of all of the multiple lighting dots 21 on the circuit board layer 20 are configured in such a way that the collection of light emitted by all of the multiple lighting dots 21 passes through the body 31 of the lower helmet body 30 and shines uniformly on the surface of a user's head.

Preferably, the circuit board layer 20 has at least 72 sets of light-emitting units.

In another embodiment, the lower helmet body 30 has multiple through holes (not shown in drawings), and thus the lower helmet body 30 needs not to be made of transparent materials. The locations of the multiple through holes on the body 31 of the lower helmet body 30 and the locations of the multiple lighting dots 31 on the circuit board layer 20 are configured in such a way that under the situation that the upper helmet body 10, the circuit board layer 20 and the lower helmet body 30 fit together, and the circuit board layer 20 is firmly fixed between the upper helmet body 10 and the lower helmet body 30, then each of the multiple lighting dots 21 is in proximity of a center position of a corresponding through hole of the body 31 of the lower helmet body 30 such that the light emitted by each of the multiple lighting dots 21 will pass through the corresponding through hole in greater proportion such as no less than 20%. The collection of light emitted by all of the multiple lighting dots 21 passes through the body 31 which has the through holes, and shines uniformly on the surface of a user's head.

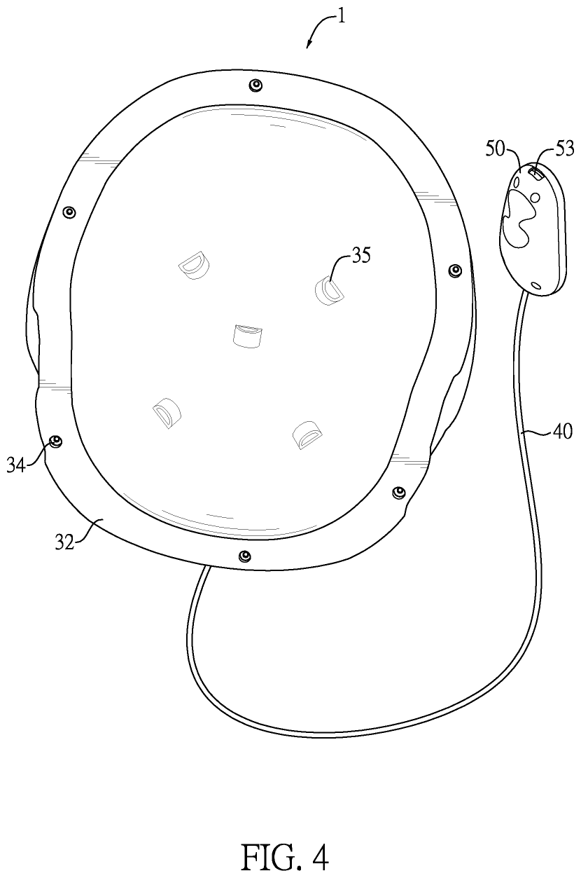

Please refer to FIG. 4. In one embodiment, the lower helmet body 30 further includes multiple cushions 35 which are fixed on the surface of the body 31 of the lower helmet body 30. The multiple cushions 35 are for separating the body 31 of the lower helmet body 30 and the user's head at least partially, so that air circulation, heat dissipation, and softness are all enhanced to greatly increase a user's comforts when he/she wears the light based therapy helmet 1 of the present invention. The multiple cushions 35 are made of plastic, leather, Styrofoam, sponge, foam, paper, bamboo, rattan, grass, wood, metal or rubber.

Based on the technical contents of the light based therapy helmet disclosed in the present invention, it is clear that a mixture of the three light bands, which are red light, infrared light, and far infrared light, enhances healthful effects. Moreover, the modularized concise structural design and ease of assembling for the upper helmet body, the circuit board layer and the lower helmet body can reduce manufacturing costs and provide users with a more comfortable, more effective and more reasonably priced device that promotes healthy hair growth, therefore achieving the purposes of the present invention.

The aforementioned are preferred embodiments of the present invention. It should be noted that for those of ordinary skill in the art, without departing from the principles of the present invention, certain improvements and retouches of the present invention can still be made which are nevertheless considered as within the protection scope of the present invention.

Even though numerous characteristics and advantages of the present invention have been set forth in the foregoing description, together with details of the structure and function of the invention, the disclosure is illustrative only. Changes may be made in detail, especially in matters of shape, size, and arrangement of parts within the principles of the invention to the full extent indicated by the broad general meaning of the terms in which the appended claims are expressed.

1. A light based therapy helmet, including:
a circuit board layer, which includes:
a circuit board; and
multiple lighting dots, fixed on the circuit board;
wherein each of the multiple lighting dots is respectively a first light-emitting unit, a second light-emitting unit or a third light-emitting unit, and the first light-emitting unit emits light of a first light band, the second light-emitting unit emits light of a second light band, and the third light-emitting unit emits light of a third light band;
a lower helmet body, which includes:
a body; and
an accommodation space, configured for a user's head to be accommodated therein directly, wherein the accommodation space is defined by the body of the lower helmet body;
an upper helmet body, which includes:
an accommodation space, which fits the circuit board layer and the lower helmet body;
wherein when the lower helmet body, the circuit board layer, and the upper helmet body fit together, the circuit board layer is fixed between the upper helmet body and the lower helmet body;
a controller, which controls ON/OFF states and emission brightness of the multiple lighting dots;
a set of interface wires connected between the circuit board layer and the controller;
wherein the body of the lower helmet body is made of transparent materials, and locations of the first light-emitting unit, the second light-emitting unit and the third light-emitting unit on the circuit board layer are configured in such a way that a collection of the respective light emitted from all of the first light-emitting units, all of the second light-emitting units, and all of the third light-emitting units passes the body of the lower helmet body respectively and shines uniformly respectively; and
wherein the light of the first light band, the light of the second light band and the light of the third light band are respectively red light, infrared light and far infrared light, and include respectively a wavelength of 660 nm, a wavelength of 940 nm and a wavelength of 1600 nm.

2. The light based therapy helmet as claimed in claim 1, wherein
a number of the first light-emitting unit, a number of the second light-emitting unit and a number of the third light-emitting unit are each no less than 24.

3-4. (canceled)

5. The light based therapy helmet as claimed in claim 1, wherein the body of the lower helmet body is made of transparent materials, and respective average transparencies of the transparent materials with respect to the light of the first light band, the light of the second light band and the light of the third light band are no less than 5%.

6. A light based therapy helmet, including
a circuit board layer, which includes:
a circuit board;
multiple lighting dots, which are fixed on the circuit board,
wherein each of the multiple lighting dots is a set of light-emitting units, and the set of light-emitting units includes a first LED which emits light of a first light band, a second LED which emits light of a second light band and a third LED which emits light of a third light band;
a lower helmet body, which includes:
a body; and
an accommodation space, configured for a user's head to be accommodated therein directly;
an upper helmet body, which includes:
an accommodation space, which fits the circuit board layer and the lower helmet body;
wherein when the lower helmet body, the circuit board layer, and the upper helmet body fit together, the circuit board layer is fixed between the upper helmet body and the lower helmet body;
a controller, which controls ON/OFF states and emission brightness of the multiple lighting dots;
a set of interface wires, having two ends, one end of which connects the circuit board layer, and the other end of which connects the controller;
wherein the body of the lower helmet body is made of transparent materials, and locations of the multiple lighting dots on the circuit board layer are configured in such a way that a collection of the light emitted from all of the multiple lighting dots passes the body of the lower helmet body and shines uniformly; and
wherein the light of the first light band, the light of the second light band and the light of the third light band are respectively red light, infrared light and far infrared light, and include respectively a wavelength of 660 nm, a wavelength of 940 nm and a wavelength of 1600 nm.

7. The light based therapy helmet as claimed in claim 6, wherein the number of the multiple lighting dots is no less than 72.

8-9. (canceled)

10. The light based therapy helmet as claimed in claim 6, wherein the body of the lower helmet body is made of transparent materials, and respective average transparencies of the transparent materials with respect to the light of the first light band, the light of the second light band and the light of the third light band are no less than 5%.

11. The light based therapy helmet as claimed in claim 1, wherein the body of the lower helmet body has multiple through holes, and the light emitted by each of the multiple lighting dots passes through a respective one among the multiple through holes, and locations of the multiple lighting dots on the circuit board layer are configured in such a way that a collection of light emitted by the multiple lighting dots passes the multiple through holes and shines uniformly.

12. The light based therapy helmet as claimed in claim 6, wherein the body of the lower helmet body has multiple through holes, and the light emitted by each of the multiple lighting dots passes through a respective one among the multiple through holes, and locations of the multiple lighting dots on the circuit board layer are configured in such a way that a collection of light emitted by the multiple lighting dots passes the multiple through holes and shines uniformly.

13. The light based therapy helmet as claimed in claim 1, wherein the controller further includes:
at least one on-off button to turn on/off the multiple lighting dots on the circuit board layer;
at least one brightness control button to control the brightness of a collection of light emitted by the multiple lighting dots.

14. The light based therapy helmet as claimed in claim 6, wherein the controller further includes:
at least one on-off button to turn on/off the multiple lighting dots on the circuit board layer;
at least one brightness control button to control the brightness of a collection of light emitted by the multiple lighting dots.

15. The light based therapy helmet as claimed in claim 1, wherein the controller further includes:
a power input end to receive power from an external power source.

16. The light based therapy helmet as claimed in claim 6, wherein the controller further includes:
a power input end to receive power from an external power source.

17. The light based therapy helmet as claimed in claim 1, wherein a type of connections configured among the circuit board layer, the set of interface wires and the controller is compatible with Universal Serial Bus Type-C cables and connectors.

18. The light based therapy helmet as claimed in claim 6, wherein a type of connections configured among the circuit board layer, the set of interface wires and the controller is compatible with Universal Serial Bus Type-C cables and connectors.

19. The light based therapy helmet as claimed in claim 1, wherein the lower helmet body further includes multiple cushions which are fixed on an inner surface of the body of the lower helmet body.

20. The light based therapy helmet as claimed in claim 6, wherein the lower helmet body further includes multiple cushions which are fixed on an inner surface of the body of the lower helmet body.
